(19) United States
(12) Patent Application Publication
(10) Pub. No.: US 2002/0065240 A1
(43) Pub. Date: May 30, 2002

(54) Gene therapy for stimulation of angiogenesis

(75) Inventors: Kenneth A. Thomas JR., Chatham Borough, NJ (US); Richard L. Kendall, Thousand Oaks, CA (US); Andrew J. Bett, Lansdale, PA (US); William R. Huckle, Blacksburg, VA (US)

Correspondence Address: MERCK AND CO INC, P O BOX 2000, RAHWAY, NJ, 070650907

(73) Assignee: Merck & Co., Inc., Rahway, NJ

(21) Appl. No.: 09/812,133

(22) Filed: Mar. 19, 2001

Related U.S. Application Data
(63) Continuation of application No. 09/530,467, filed on Apr. 26, 2000, now abandoned.
Application No. 09/530,467 is a 371 of international application No. PCT/US98/22668, filed on Oct. 23, 1998.
(60) Provisional application No. 60/063,629, filed on Oct. 27, 1997.

(51) Int. Cl. A61K 48/00; C12N 7/01

(52) U.S. Cl. 514/44; 424/93.21; 435/235.1

(57) ABSTRACT

The present invention relates to methods of gene therapy to promote angiogenesis in the treatment of peripheral, cardiac and other pathological tissue ischemias utilizing a DNA molecule (SEQ ID NO:1) which encodes human VEGF₁₄₅, set forth in SEQ ID NO:2.

### CROSS-REFERENCE TO RELATED APPLICATIONS

Not applicable

### STATEMENT REGARDING FEDERALLY-SPONSORED R&D

Not applicable.

### REFERENCE TO MICROFICHE APPENDIX

Not applicable.

### FIELD OF THE INVENTION

The present invention relates to methods of gene therapy to promote angiogenesis in the treatment of peripheral, cardiac and other pathological tissue ischemias utilizing a nucleotide sequence which encodes VEGF₁₄₅.

### BACKGROUND OF THE INVENTION

Vascular endothelial cells form a luminal non-thrombogenic monolayer throughout the vascular system. Mitogens promote embryonic vascular development, growth, repair and angiogenesis in these cells. Angiogenesis involves the proteolytic degradation of the basement membrane on which endothelial cells reside followed by the subsequent chemotactic migration and mitosis of these cells to support sustained growth of a new capillary shoot. One class of mitogens selective for vascular endothelial cells include vascular endothelial growth factor (referred to as VEGF or VEGF-A) and the homologues placenta growth factor (PlGF), VEGF-B, VEGF-C and VEGF-D.

Human VEGF exists as a glycosylated homodimer in one of five mature processed forms containing 206, 189, 165, 145 and 121 amino acids, the most prevalent being the 165 amino acid form.

U.S. Pat. No. 5,240,848 discloses the nucleotide and amino acid sequence encoding the 189 amino acid form of human VEGF.

U.S. Pat. No. 5,332,671 discloses the nucleotide and amino acid sequence encoding the 165 amino acid form of human VEGF.

Charnock-Jones et al (1993, Biol. Reproduction 48: 1120-1128) and Sharkey et al (1993, J. Reprod. Fertility 99, 609-615) disclose a human VEGF₁₄₅ splice variant mRNA. Cheung et al. (1995, Am. J. Obstet. Gynecol. 173, 753-759) disclose an ovine VEGF145 splice variant mRNA. These disclosures do not demonstrate the presence or activity of the protein product.

Poltorak et al. (1997, J. Biol. Chem. 272, 7151-7158) disclosed that recombinant human VEGF₁₄₅ has approximately one-sixth activity as a mitogen when compared to recombinant human VEGF₁₆₅.

U.S. Pat. No. 5,194,596 discloses the nucleotide and amino acid sequence encoding the 121 amino acid form of human VEGF.

The 206 amino acid and 189 amino acid forms of human VEGF each contain a highly basic 24-amino acid insert that promotes tight binding to heparin, and presumably, heparin proteoglycans on cellular surfaces and within extracellular matrices (Ferrara, et al., 1991, J. Cell. Biochem. 47: 211-218). The VEGF₁₆₅ form binds heparin to a lesser extent while VEGF₁₂₁ does not bind heparin.

Human PlGF is also a glycosylated homodimer which shares 46% homology with VEGF at the protein level. Differential splicing of human PlGF mRNA leads to either a 170 amino acid or 149 amino acid precursor, which are proteolytically processed to mature forms of 152 or 131 amino acids in length, respectively (Bayne and Thomas, EP Publication #0506477 [Sep. 30, 1992]; Maglione, et al., 1993, Oncogene 8: 925-931; Hauser and Weich, 1993, Growth Factors 9: 259-268).

VEGF-B has been isolated and characterized (Grimmond et al., 1996, Genome Research 6: 124-131; Olofsson et al., 1996, Proc. Natl. Acad. Sci. USA 93: 2576-2581). The fill-length human cDNAs encode 188 and 207 amino acid residue precursors wherein the NH₂ terminal portions are proteolytically processed to mature forms 167 and 186 amino acid residues in length. Human VEGF-B expression was found predominantly in heart and skeletal muscle as a disulfide-linked homodimer. However, human VEGF-B may also form a heterodimer with VEGF (id. @ 2580).

VEGF-C has also been isolated and characterized (Joukov et al., 1996, EMBO J. 15: 290-298; see also PCT International application WO 96/39515). A cDNA encoding VEGF-C was obtained from a human prostatic adenocarcinoma cell line. A 32 kDa precursor protein is proteolytically processed to generate the mature 23 kDa form, which binds the receptor tyrosine linase, Flt4.

VEGF-D was identified in an EST library, the full-length coding region was cloned and recognized to be most homologous to VEGF-C among the VEGF family amino acid sequences (Yamada, et al., 1997, Genomics 42:483488). The human VEGF-D mRNA was shown to be expressed in lung and muscle.

VEGF and its homologues impart activity by binding to vascular endothelial cell plasma membrane-spanning tyrosine kinase receptors which then activate signal transduction and cellular signals. The Flt receptor family is a major tyrosine kinase receptor which binds VEGF with high affinity. At present the flt receptor family includes flt-1 (Shibuya, et al., 1990, Oncogene 5: 519-524), KDR/flk-1(Terman, et al., 1991, Oncogene 6: 1677-1683; Terman, et al., 1992, Biochem. Biophys. Res. Commun. 187: 1579-1586), and flt-4 (Pajusola, et al., 1992, Cancer Res. 52: 5738-5743).

Vascular endothelial growth factor (VEGF) binds the high affinity membrane-spanning tyrosine kinase receptors KDR and Flt-1. Cell culture and gene knockout experiments indicate that each receptor contributes to different aspects of angiogenesis. KDR mediates the mitogenic function of VEGF whereas Flt-1 appears to modulate non-mitogenic functions perhaps including cellular adhesion and/or migration. Inhibiting KDR thus significantly diminishes the level of mitogenic VEGF activity.

Isner et al. (1996, The Lancet 348:370-374) disclose that administration of a DNA plasmid vector encoding recombinant human VEGF₁₆₅ to a human patient improved blood supply to an ischemic limb.

Despite recent advances in identifying genes encoding ligands and receptors involved in angiogenesis, there is no indication that gene therapy based on delivery and expression of VEGF₁₄₅ would promote the level of angiogenesis required to overcome peripheral or cardiac ischemias. The present invention addresses and meets this need.

### SUMMARY OF THE INVENTION

The present invention relates to methods of gene therapy for stimulating VEGF-induced angiogenesis associated with ischemic peripheral and/or cardiac muscle. Vascular endothelial growth factor acts as a mitogen to stimulate local angiogenesis from vascular endothelial cells so as to increase neovascularization, perfusion and performance of ischemic peripheral and/or cardiac muscle. A nucleic acid molecule encoding VEGF₁₄₅ or mutant versions thereof may be delivered either systemically or locally in a direct manner to target cells of the mammalian host by viral or non-viral based methods. A preferred mammalian host of the present invention is a human.

The present invention therefore relates to gene transfer of a nucleic acid molecule and concomitant in vivo expression of a soluble form of a mammalian VEGF₁₄₅ protein within a mammalian host. It is preferred that the form of VEGF used to practice the present invention be a mammalian splice variant related to human VEGF₁₄₅. An especially preferred form for use in gene therapy application of the present invention is a DNA molecule encoding human VEGF₁₄₅. It will be within the purview of the skilled artisan to generate one or more alternative forms of human VEGF₁₄₅, a form which promotes angiogenesis on par with other forms of mammalian VEGF, and especially on par with other human VEGF forms, including but not limited to human VEGF₁₈₉, human VEGF₁₆₅, human VEGF₁₂₁, human VEGF-B, human VEGF-C and human VEGF-D. Such a VEGF₁₄₅ gene therapy vehicle may be generated by recombinant DNA techniques known in the art using a DNA fragment encoding a partial or complete amino acid sequence of human VEGF₁₄₅. Using recombinant DNA techniques, DNA molecules are constructed which encode at least a portion of human VEGF₁₄₅ receptor capable of stimulating angiogenesis. Standard recombinant DNA techniques are used such as those found in Maniatis, et al. (1982, Molecular Cloning: A Laboratory Manual; Cold Spring Harbor Laboratory, Cold Spring Harbor, N.Y.) and which are exemplified within the confines of the specification.

In an especially preferred embodiment of the present invention, a DNA molecule comprising the nucleotide sequence as set forth in SEQ ID NO:1 is a template for constructing a gene therapy vector. Such a gene therapy vector will express human VEGF₁₄₅ (SEQ ID NO:2) or a biologically active form of human VEGF₁₄₅ promotes angiogenesis subsequent to delivery to a mammalian host in order to combat cardiac or peripheral ischemia.

In another especially preferred embodiment of the present invention, a DNA molecule which encodes the human VEGF₁₄₅ protein as set forth in SEQ ID NO:2, or a biologically active form, is a template for constructing a gene therapy vector. Such a gene therapy vector will express human VEGF₁₄₅ (SEQ ID NO:2) or a biologically active form of human VEGF₁₄₅ and promote angiogenesis subsequent to delivery to a mammalian host in order to combat cardiac or peripheral ischemia.

Any VEGF₁₄₅ construct, including but not necessarily limited to a human VEGF₁₄₅ construct comprising the DNA sequence as set forth in SEQ ID NO:1, and biologically active form thereof, may be delivered to a mamalian host using a vector or other delivery vehicle. A DNA fragment encoding VEGF₁₄₅ or biologically active mutant versions thereof may be delivered either systemically or locally to target cells in the proximity of or within an ischemic tissue of a mammalian host by viral or non-viral based methods. Viral vector systems which may be utilized in the present invention include, but are not limited to, (a) adenovirus vectors; (b) retrovirus vectors; (c) adeno-associated virus vectors; (d) herpes simplex virus vectors; (e) SV 40 vectors; (f) polyoma virus vectors; (g) papilloma virus vectors; (h) picarnovirus vectors; and (i) vaccinia virus vectors. Non-viral methods of delivery include but are not necessarily limited to direct injection of naked DNA, such as any recombinant DNA plasmid expression vector described herein which comprises a DNA fragment encoding VEGF₁₄₅. Additional non-viral vectors include but are not limited to DNA-lipid complexes, for example liposome-mediated or ligand/poly-L-Lysine conjugates, such as asialoglyco-protein-mediated delivery systems.

A preferred viral vector of the present invention is a first, second or helper dependent adenovirus vector.

An especially preferred first generation recombinant Ad/VEGF₁₄₅ virus is AdVEGF145.

A preferred non-viral vector system of the present invention relates to use of a DNA plasmid expression vector, of which numerous examples are known to the skilled artisan. As noted below, an expression vector is any polynucleotide having regulatory regions operably linked to a coding region such that, when in a host cell, the vector can direct the expression of the coding sequence. The expression vectors utilized to practice the present invention will comprise regulatory regions which promote expression within the target cell so as to impart a therapeutic effect on a particular ischemia within the mammalian host.

In addition to gene therapy related applications involving nucleic acid molecules encoding VEGF₁₄₅, a VEGF₁₄₅ protein or biologically active fragment thereof may be utilized to treat various peripheral and/or cardiac ischemias in the mammalian host, preferably a human. Recombinant human VEGF₁₄₅ as exemplified within this specification may be delivered from slow release polymers or devices into ischemic tissue or systemically. Pharmaceutically useful compositions comprising VEGF₁₄₅ can be formulated according to known methods such as by the admixture of a pharmaceutically acceptable carrier. Examples of such carriers and methods of formulation can be found in Remington's Pharmaceutical Sciences. To form a pharmaceutically acceptable composition suitable for effective administration, such compositions will contain a biologically effective amount of the VEGF₁₄₅ protein, preferably recombinant human VEGF₁₄₅ protein.

As used herein, “WEGF” or “VEGF-A” refers to vascular endothelial growth factor, which comprises proteins which are translational products of various splice variants, particularly VEGF₁₂₁, VEGF₁₄₅, VEGF₁₆₅ and VEGF₁₈₉. A particular splice variant is referred to with the appropriate amino acid total of the mature form of the protein (e.g., VEGF₁₂₁).

As used herein, “homologue of VEGF” refers to homodimers of VEGF-B, VEGF-C, VEGF-D and PlGF and any functional heterodimers formed between VEGF-A, VEGF-B, VEGF-C, VEGF-D and PlGF, including but not limited to a VEGF-A/PlGF heterodimer.

As used herein, “VEGF-B” refers to vascular endothelial growth factor-B.

As used herein, “VEGF-C” refers to vascular endothelial growth factor-C.

As used herein, “VEGF-D” refers to vascular endothelial growth factor-D.

As used herein, “KDR” or “FLK-1” refers to kinase insert domain-containing receptor or fetal liver kinase.

As used herein, “FLT-1” refers to fms-like tyrosine kinase receptor.

As used herein, “Ad” refers to adenovirus.

As used herein, “HUVECs” refers to human umbilical vein endothelial cells.

As used herein, the term “mammalian host” refers to any mammal, including a human being.

As used herein, the term “hVEGF₁₄₅” refers to human VEGF₁₄₅.

As used herein a “polynucleotide” is a nucleic acid of more than one nucleotide. A polynucleotide can be made up of multiple polynucleotide units that are referred to by description of the unit. For example, a polynucleotide can comprise within its bounds a polynucleotide(s) having a coding sequence(s), a polynucleotide(s) that is a regulatory region(s) and/or other polynucleotide units commonly used in the art.

As used herein, an “expression vector” is a polynucleotide having regulatory regions operably linked to a coding region such that, when in a host cell, the vector can direct the expression of the coding sequence. The use of expression vectors is well known in the art. Expression vectors can be used in a variety of host cells and, therefore, the regulatory regions are preferably chosen as appropriate for the particular host cell.

As used herein, a “biologically active fragment”, “biologically active form”, “biologically active equivalent” or “functional derivative” of a wild-type human VEGF₁₄₅ possesses a biological activity that is at least substantially equal to the biological activity of the wild type human VEGF₁₄₅. The above-mentioned terms are intended to include “fragments”, “mutants,” or “variants,” of the wild type human VEGF₁₄₅ protein which is not substantially similar to other known VEGF homologues. The term “fragment” is meant to refer to any polypeptide subset of wild-type human VEGF₁₄₅ which is not substantially similar in structure to other known VEGF homologues. The term “mutant” is meant to refer to a molecule that may be substantially similar to the wild-type form but possesses distinguishing biological characteristics. Such altered characteristics include but are in no way limited to altered substrate binding, altered substrate affinity and altered sensitivity to chemical compounds affecting biological activity of the human VEGF₁₄₅ or human VEGF₁₄₅ functional derivative which may make the respective mutant attractive for the gene therapy applications disclosed within the confines of this specification. The term “variant” is meant to refer to a molecule substantially similar in structure and function to either the entire wild-type protein or to a fragment thereof.

It is an object of the present invention to provide systemic or localized delivery of VEGF₁₄₅ to a mammalian host, and preferably a human host, to stimulate angiogenesis for treatment of peripheral and cardiac ischemia.

It is also an object of the present invention to utilize a gene or gene fragment of human VEGF₁₄₅ in gene therapy methods to stimulate angiogenesis for treatment of peripheral and cardiac ischemia.

It is an object of the present invention to provide recombinant DNA vectors containing VEGF₁₄₅ constructs, preferably human VEGF₁₄₅ constructions, for use in gene therapy to stimulate angiogenesis for treatment of peripheral and cardiac ischemia.

### BRIEF DESCRIPTION OF THE DRAWINGS

FIG. 1 shows the cDNA sequence which encodes the full-length translation product precursor of mature human VEGF₁₄₅. The TGA translation termination codon is shown and the sequence is grouped by codon.

FIG. 2 shows the amino acid sequence of human VEGF₁₄₅ in single letter code, and as set forth in SEQ ID NO:2 in three letter code. The deduced amino acid sequence of the full-length translation product includes the predicted N-terminal 26 amino acid residue secretory leader sequence (underlined).

FIG. 3 shows reversed-phase C4 Chromatography of pooled VEGF₁₄₅. The pooled VEGF₁₄₅-containing sample from the Mono S column was loaded onto a C4 reversed phase HPLC column equilibrated in 0.1% acetonitrile and eluted at flow rate of 200 μl/min with a 0-70% (v/v) gradient of acetonitrile monitoring absorbance at 215 nm.

FIG. 4 shows a Western blot of hVEGF₁₄₅. Fractions 15 and 16 from the C4 HPLC chromatographic fractionation were analyzed by SDS/PAGE and Western blotting using an anti-VEGF antibody. The positions and masses in kDa of molecular mass markers are denoted on the right of the figure.

FIG. 5 shows the purity of hVEGF₁₄₅. Fraction 15 from the C4 reversed phase HPLC column was analyzed by SDS/PAGE on a 4-20% gradient gel and silver stained. The positions and masses in kDa of molecular mass markers are denoted on the right of the figure.

FIG. 6 shows VEGF₁₄₅ mitogenic activity. Purified human recombinant VEGF₁₄₅ (open circles) and VEGF₁₆₅ (filled circles) were assayed in parallel as a function of dose on human umbilical vein vascular endothelial cells in culture. Mitogenesis was monitored by incorporation of [³H]thymidine into DNA and expressed as percent maximum response. Each dose-response curve is the average of 3 separate determinations.

FIG. 7 shows DNA plasmid expression vector, V1Jns.

### DETAILED DESCRIPTION OF THE INVENTION

The present invention relates to methods of gene therapy for stimulating VEGF-induced angiogenesis including but not limited to ischemic peripheral and/or cardiac tissue. Vascular endothelial growth factor acts as a mitogen to stimulate local angiogenesis from vascular endothelial cells so as to increase neovascularization, perfusion and performance of ischemic peripheral and/or cardiac muscle. A nucleic acid molecule encoding VEGF₁₄₅ or mutant versions thereof may be delivered either systemically or locally in a direct manner to target cells of the mammalian host by viral or non-viral based methods.

The present invention therefore relates to gene transfer of a nucleic acid molecule and concomitant in vivo expression of a soluble form of a mammalian VEGF₁₄₅ protein within a mammalian host. It is preferred that the form of VEGF used to practice the present invention be a mammalian splice variant related to human VEGF₁₄₅. An especially preferred form for use in gene therapy application of the present invention is a DNA molecule encoding human VEGF₁₄₅.

Therefore, the present invention relates in part to fully active human VEGF₁₄₅ to be delivered as a protein or gene therapeutic agent to promote angiogenesis. The cDNA fragment which encodes human VEGF₁₄₅ (as set forth in SEQ ID NO:1) was generated by PCR-based hybridization to human placenta total RNA, cDNA synthesis, and isolation of a positive clone for further characterization. The corresponding hVEGF₁₄₅ protein has been expressed from a baculovirus expression system in insect cells and chromatographically purified to homogeneity. Therefore, the present invention provides for expression of fully active recombinant VEGF₁₄₅ protein either in culture or by in vivo gene transfer delivery from plasmids or viral vectors to promote hVEGF₁₄₅ expression and to promote angiogenesis in and around ischemic tissue. Messenger RNA encoding a human alternatively spliced isoform of vascular endothelial growth factor (VEGF) that contains ₁₄₅ amino acids in the mature processed form had been identified but the native protein was not demonstrated to exist so its effective translation and expression as a stably folded and active protein was not established. It is known that expression is not guaranteed by the presence of mRNA as shown in the case of several mRNAs encoding IGF II. A recombinant form of VEGF₁₄₅ was reported to be only ⅙th as active as VEGF165 (Poltorak et al., 1997, J. Biol. Chem. 272: 7151-7158). Therefore, the disclosed gene therapy applications of the present invention are exemplified in part by showing that recombinant human VEGF₁₄₅ is equivalently active to human VEGF₁₆₅. This data demonstrates, in stark contrast to the above-mentioned publication, that hVEGF₁₄₅ is a fully functional VEGF isoform. As a consequence of the full mitogenic activity of VEGF₁₄₅, it is now established within the confines of this specification that VEGF₁₄₅ is an appropriate gene/protein for use as a therapeutic agent as indicated herein.

To this end, a particular embodiment of the present invention involves the use of a human recombinant form of VEGF₁₄₅ in gene therapy protocols. It will be within the purview of the skilled artisan to generate additional forms of human VEGF₁₄₅ which are within the scope of the present invention. Any such biologically active alternative form will be structurally similar to VEGF₁₄₅ when compared to known VEGF homologues and will promote angiogenesis at a substantially similar level as with other such mammalian VEGF homologues, and especially at a substantially similar level as human VEGF homologues, including but not limited to human VEGF₁₈₉, human VEGF₁₆₅, human VEGF₁₂₁, human VEGF-B, human VEGF-C and human VEGF-D. Such a VEGF₁₄₅ gene therapy vehicle may be generated by recombinant DNA techniques known in the art using a DNA fragment encoding a complete or partial amino acid sequence of human VEGF₁₄₅. Again, any such partial or fragmented version of VEGF₁₄₅ will be substantially more similar to VEGF₁₄₅ as compared to other known forms of VEGF. Using recombinant DNA techniques, DNA molecules are constructed which encode at least a portion of human VEGF₁₄₅ receptor capable of stimulating angiogenesis. Standard recombinant DNA techniques are used such as those found in Maniatis, et al. (1982, Molecular Cloning: A Laboratory Manual; Cold Spring Harbor Laboratory, Cold Spring Harbor, N.Y.).

In a preferred embodiment of the present invention, a DNA fragment comprising the nucleotide sequence as set forth in SEQ ID NO:1 encoding human VEGF₁₄₅ is the template for constructing a gene therapy vector wherein expressed human VEGF₁₄₅ (SEQ ID NO:2) or a biologically active form promotes angiogenesis subsequent to delivery to a mammalian host in order to offer therapeutic treatment for cardiac or peripheral ischemia. Therefore, the present invention discloses methods of gene therapy utilizing a DNA molecule encoding VEGF₁₄₅ (preferably a DNA molecule encoding hVEGF₁₄₅) and DNA molecules encoding biologically active fragments as noted herein, or various pharmaceutical applications of VEGF₁₄₅ protein (preferably hVEGF₁₄₅ protein) and biologically active fragments herein, to increase neovascularization, perfusion and performance of ischemic peripheral and cardiac muscle.

Briefly, a cDNA clone encoding human VEGF₁₄₅ was isolated by PCR-mediated screening of a human placental total RNA. The human placenta total RNA was used for the first strand cDNA. The reaction was primed with random hexamers. Following the synthesis reaction aliquots (2 μl) were used as templates in polymerase chain reactions using Pfu DNA polymerase. The following primers derived from the rat cDNA sequence were used for the amplification of human VEGFs: forward primer, 5′ACGGGATCCAAATATGAACTTTCTGCTCTCTTG-3′ (SEQ ID NO:3); reverse primer, 5′-TGGAAGCTTTCACCGCCTTGGCTTGTC-3′ (SEQ ID NO:4). Each primer contains a single nucleotide base change when compared to the isolated human DNA sequence. However the predicted protein sequence is identical to the human amino acid sequence (i.e., codon CTC→CTG for Pro as amino acid #5 of the signal sequence of VEGF₁₄₅ for the forward primer and codon CCA→CCG for Arg as amino acid #143 of mature form of VEGF145 for the reverse primer) as predicted from known sequence analysis and splice variant analysis of human VEGF homologues. PCR products were visualized by gel electrophoresis and a band corresponding to the expected size of VEGF₁₄₅ was detected only in the placental RNA reaction. The appropriate sized cDNA molecules were subcloned into a pCR-blunt plasmid, which was transfected into competent E. coli cells, grown, isolated and digested with EcoRI. Clones with the insert size expected for the human VEGF145 gene fragment were confirmed by DNA sequence analysis.

A DNA fragment encoding human VEGF₁₄₅, set forth as SEQ ID NO:1, is as follows:

ATG AAC TTT CTG CTC TCT TGG GTG CAT TGG
AGC CTT GCC TTG CTG CTC TAG CTC GAC CAT
GCC AAG TGG TCC CAG GCT GCA CCC ATC GCA
GAA GGA GGA GGG CAG AAT CAT GAG GAA GTG
GTG AAG TTC ATG GAT GTC TAT GAG CGC AGC
TAC TGC CAT CCA ATC GAG ACC CTG GTG GAG
ATC TTC CAG GAG TAC CCT GAT GAG ATC GAG
TAC ATC TTC AAG CGA TCG TGT GTG CCC GTG
ATG CGA TGC GGG GGC TGC TGC AAT GAC GAG
GGC CTG GAG TGT GTG CCC ACT GAG GAG TCC
AAC ATC ACC ATG CAG ATT ATG GGG ATC AAA
CCT CAC CAA GGC CAG CAC ATA GGA GAG ATC
AGC TTC CTA GAG CAC AAC AAA TCT GAA TGC
AGA CCA AAG AAA CAT AGA GCA AGA CAA GAA
AAA AAA TCA GTT CGA GGA AAG GGA AAG GGG
CAA AAA CGA AAG CGC AAG AAA TCC CGG TAT
AAG TCC TGG AGC GTG TCT GAC AAG CCA AGG
CGG TGA (SEQ ID NO:1).

The human recombinant VEGF₁₄₅ protein expressed from SEQ ID NO:1 is set forth as SEQ ID NO:2 and is as follows:

MNFLLSWVHW SLALLLYLHH AKWSQAAPMA
EGGGQNHHEV VKFMDVYQRS YCHPIETLVD IFQEYPDEIE
YIFKPSCVPL MRCGGGCNDE GLECVPTEES NITMQIMRIK
PHQGQHIGEM SFLQHNKCEC RPKKDRARQE KKSVRGKGKG
QKRKRKKSRY KSWSVCDKPR R (SEQ ID NO:2),

as shown in single letter code. The underlined portion represents the putative signal peptide for human VEGF_{145.}

Expression vectors are defined herein as DNA sequences that are required for the transcription of cloned copies of genes and the translation of their mRNAs in an appropriate host. Such vectors can be used to express eukaryotic genes in a variety of hosts such as bacteria, bluegreen algae, fungal cells, yeast cells, plant cells, insect cells and animal cells.

Specifically designed vectors allow the shuttling of DNA between hosts such as bacteria-yeast or bacteria-animal or bacteria-insect cells. An appropriately constructed expression vector should contain: an origin of replication for autonomous replication in host cells, selectable markers, a limited number of useful restriction enzyme sites, a potential for high copy number, and active promoters. A promoter is defined as a DNA sequence that directs RNA polymerase to bind to DNA and initiate RNA synthesis. A strong promoter is one which causes mRNAs to be initiated at high frequency. Expression vectors may include, but are not limited to, cloning vectors, modified cloning vectors, specifically designed plasmids or viruses.

One embodiment of the present invention relates to a non-viral vector which is a recombinant plasmid vector comprising a nucleotide sequence encoding VEGF₁₄₅. A preferred aspect of this embodiment is a recombinant plasmid vector which comprises a nucleotide fragment which comprises human VEGF₁₄₅ as set forth in SEQ ID NO:1. It will be within the purview of the artisan of ordinary skill to pick and choose between available recombinant expression plasmids which express human VEGF₁₄₅ at therapeutically acceptable levels within the mammalian host.

In another especially preferred embodiment of the present invention, a DNA molecule which encodes the human VEGF₁₄₅ protein as set forth in SEQ ID NO:2, or a biologically active form, is a template for constructing a gene therapy vector. Such a gene therapy vector will express human VEGF₁₄₅ (SEQ ID NO:2) or a biologically active form of human VEGF₁₄₅ and promote angiogenesis subsequent to delivery to a mammalian host in order to combat cardiac or peripheral ischemia.

DNA encoding VEGF₁₄₅ or a biologically active fragment as defined herein may also be cloned into an expression vector for expression in a recombinant host cell. Recombinant host cells may be prokaryotic or eukaryotic, including but not limited to bacteria, yeast, mammalian cells including but not limited to cell lines of human, bovine, porcine, monkey and rodent origin, and insect cells including but not limited to drosophila, moth, mosquito and armyworm derived cell lines. The expression vector may be introduced into host cells via any one of a number of techniques including but not limited to transformation, transfection, Ad/polylysine DNA complexes, protoplast fusion, and electroporation. Cell lines derived from mammalian species which may be suitable and which are commercially available, include but are not limited to, CV-1 (ATCC CCL 70), COS-1 (ATCC CRL 1650), COS-7 (ATCC CRL 1651), CHC-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26) and MRC-5 (ATCC CCL 171) and HEK 293 cells. Insect cell lines which may be suitable and are commercially available include but are not limited to 3M-S (ATCC CRL 8851) moth (ATCC CCL 80) mosquito (ATCC CCL 194 and 195; ATCC CRL 1660 and 1591) and armyworm (Sf9, ATCC CRL 1711) and Sf21 (Invitrogen).

Commercially available mammalian expression vectors which may be suitable for recombinant human VEGF₁₄₅ expression include but are not limited to, pcDNA3.1 (Invitrogen), pBlueBacHis2 or pBlue Bac 4 (Invitrogen), pLITMUS28, pLITMUS29, pLITMUS38 and pLITMUS39 (New England Bioloabs), pcDNAI, pcDNAIamp (Invitrogen), pcDNA3 (Invitrogen), pMC1neo (Stratagene), pXT1 (Stratagene), pSG5 (Stratagene), EBO-pSV2-neo (ATCC 37593) pBPV-1(8-2) (ATCC 37110), pdBPV-MMTneo(342-12) (ATCC 37224), pRSVgpt (ATCC 37199), pRSneo (ATCC 37198), pSV2-dhrf (ATCC 37146), pUCTag (ATCC 37460), and lZD35 (ATCC 37565).

The cloned human VEGF₁₄₅ cDNA obtained through the methods described above may be recombinantly expressed by molecular cloning into an expression vector containing a suitable promoter and other appropriate transcription regulatory elements, and transferred into prokaryotic or eukaryotic host cells to produce recombinant VEGF₁₄₅. Techniques for such manipulations are fully described in Maniatis, et al.(id.), and are well known in the art. As a example, and not as a limitation, the VEGF₁₄₅ cDNA as set forth in SEQ ID NO:1 was expressed in a Baculovirus expression system for the purpose of obtaining purified preparations of hVEGF₁₄₅ and to test the ability of this protein to stimulate vascular endothelial cell mitogenesis necessary for angiogenesis. The hVEGF₁₄₅ gene fragment was isolated as a Bam HI/Hind III fragment and subsequently subcloned into the baculovirus expression vector pBlueBac4. Human VEGF₁₄₅ protein was expressed in Sf21 cells and concentrated through a heparin-Sepharose column. Human VEGF₁₄₅ fractions were identified loaded onto a Mono S HR5/5 column. Peak fractions were pooled, loaded onto and eluted from a C4 column. Human VEGF₁₄₅ was identified by Western blot and the purity was determined by separation on a 4-20% SDS/PAGE gel and then visualized by silver stain.

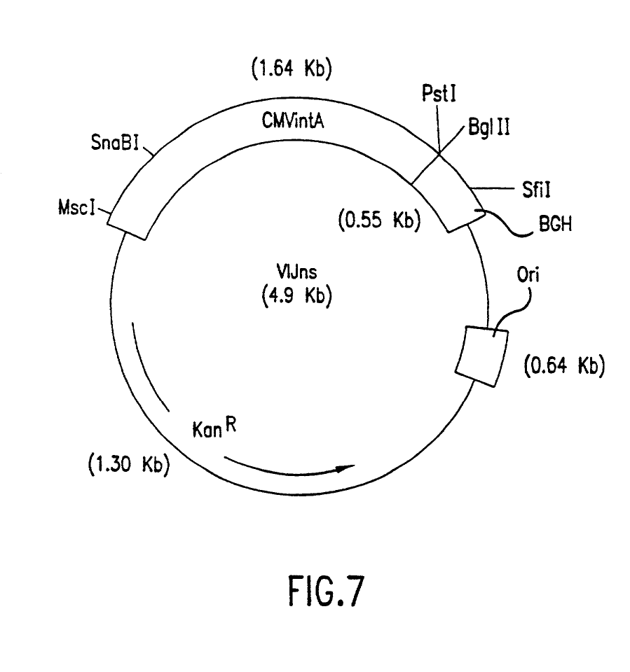

Additional expression vector and modifications thereof may be utilized which have been optimized for polynucleotide vaccinations. Essentially all extraneous DNA is removed, leaving the essential elements of transcriptional promoter, transcriptional terminator, bacterial origin of replication and antibiotic resistance gene. As noted throughout this specification, standard techniques of molecular biology for preparing and purifying DNA constructs enable the preparation of various DNA plasmid expression vectors. Numerous expression vectors which may be utilized to practice the gene therapy applications of the present invention are described in full within PCT International Application WO97/31115, which is hereby incorporated by reference. For example, V1Jns is a DNA plasmid expression vector which comprises a CMV immediate-early (IE) promoter, bovine growth hormone (BGH) polyadenylation site, and a pUC backbone. It is also possible to replace the wild type signal sequence of VEGF₁₄₅ with a signal sequence from another protein, such as but not limited to tissue-specific plasminogen activator (tPA) gene, resulting in V1Jns-tPA. Additional DNA plasmid vectors described within WO97/31115 which may be used to practice the present invention in addition to V1Jns and V1Jns-tPA, includes but is not limited to V1Jneo. The nucleotide sequence of expression plasmid V1Jneo is set forth as SEQ ID NO:5. The expression plasmid V1Jns was constructed by introducing an Sfi I site into V1Jneo. A commercially available 13 base pair Sfi I linker (New England BioLabs) was added at the Kpn I site within the BGH sequence of the vector. V1Jneo was linearized with KpnI, gel purified, blunted by T4 DNA polymerase, and ligated to the blunt Sfi I linker. Clonal isolates were chosen by restriction mapping and verified by sequencing through the linker. The map of expression vector V1Jns is shown in FIG. 7. To this end, a preferred, but in no way limiting plasmid expression vector which encodes human VEGF145 is pV1JnsVEGF145, which is constructed as follows: A pCR-blunt clone described in Example Section 1 which comprises VEGF₁₄₅ (pCR-VEGF₁₄₅-1) is digested with BamHI and EcoRI and ligated into BamHI/EcoRI digested pV1Jns, which will generate V1JnsVEGF145.

It is shown in Example Section 3 that purified recombinant human VEGF₁₄₅ stimulate proliferation of HUVEC monolayers in culture. It is known that expression of VEGF mitogenic receptors that mediate mitogenic responses to the growth factor is largely restricted to vascular endothelial cells. Human umbilical vein endothelial cells in culture proliferate in response to VEGF treatment and can be used as an assay system to quantify the effects VEGF isoforms. In the assay described in Example Section 3, quiescent HUVEC monolayers are stimulated to proliferate upon addition of VEGF. Purified baculovirus-expressed human VEGF₁₄₅ was filly efficacious as an endothelial cell mitogen when compared to baculovirus-expressed human VEGF₁₆₅ with equivalent half-maximal activities of 17-19 ng/ml for VEGF₁₆₅ and VEGF₁₄₅.

Therefore, any VEGF₁₄₅ construct, including but not necessarily limited to a human VEGF₁₄₅ construct comprising the DNA molecule as set forth in SEQ ID NO:1 or a DNA molecule which encodes the human VEGF₁₄₅ protein as set forth in SEQ ID NO:2, and biologically active forms thereof, may be delivered to the mammalian host using a vector or other delivery vehicle. As noted elsewhere in this specification, the preferred host of the present invention is a human host. In addition to a DNA plasmid expression vector as a DNA delivery vector for VEGF₁₄₅-based gene therapy, other non-viral DNA delivery vehicles include but are not limited to DNA-lipid complexes, for example liposome-mediated or ligand/poly-L-Lysine conjugates, such as asialoglyco-protein-mediated delivery systems (see for example: Felgner et al., 1994, J. Biol. Chem. 269:2550-2561; Derossi et al., 1995, Restor. Neurol. Neuros. 8:7-10; and Abcallah et al., 1995, Biol. Cell 85:1-7). It is preferred that local cells such as muscle cells be targeted for delivery and concomitant in vivo expression of the respective VEGF₁₄₅ protein to promote angiogenesis in and around the damaged tissue. A viral or non-viral recombinant gene therapy vehicle comprising a DNA fragment encoding VEGF₁₄₅ or mutant versions thereof may be delivered either systemically or locally to the target tissue and/or tissue adjacent to the ischemic region. However, other modes of administration of non-viral gene therapy vehicles are contemplated for this portion of the invention, including but not necessarily limited to subcutaneous, topical, oral, and intraperitoneal administration, all using forms well known to those of ordinary skill in the pharmaceutical arts.

Other DNA delivery vehicles include viral vectors such as adenoviruses, adeno-associated viruses, retroviral vectors (see, for example: Chu et al., 1994, Gene Therapy 1: 292-299; Couture et al., 1994, Hum. Gene Therapy. 5:, 667-277; and Eiverhand et al., 1995, Gene Therapy 2:336-343), or a combination system such as a recombinant chimeric adenoviral/retroviral vector system as described by Feng et al (1997, Nature Biotechnology 15(9): 866-870).

One such embodiment of the present invention is utilization of a first or second generation recombinant adenovirus (Ad) system for systemic or local delivery of a DNA fragment encoding VEGF₁₄₅ or mutant versions thereof to the target cells of the mammalian host. A particularly useful first generation adenovirus system used to exemplify this portion of the present invention is described in Example Section 4. A first generation recombinant Ad/VEGF₁₄₅ is one preferred gene therapy vehicle for systemic or local delivery to ischemic tissue for the purpose of stimulating angiogenesis. An especially preferred recombinant Ad/VEGF₁₄₅ virus is AdVEGF145.

Another embodiment of the present invention is utilization of a helper-dependent recombinant adenovirus (Ad) system for systemic or local delivery of a DNA fragment encoding VEGF₁₄₅ or mutant versions thereof to the target cells of the mammalian A particularly useful adenovirus system used to exemplify this portion of the present invention is described in Example Section 5 and is based on the system described by Parks et al. (1996, Proc. Natl. Acad. Sci. (USA) 93:13565-13570). A helper-dependent recombinant Ad/VEGF₁₄₅ is also a preferred gene therapy vehicle for systemic or local delivery of a VEGF₁₄₅-encoding DNA fragment to ischemic tissue for the purpose of stimulating angiogenesis. An especially preferred helper-dependent recombinant Ad/VEGF₁₄₅ virus is AdHDVEGF145-1 or AdHDVEGF145-2.

The recombinant first, second or helper-dependent Ad/VEGF₁₄₅ viruses of the present invention, including but not limited to AdVEGF145 (first generation), AdHDVEGF145-1 and AdHDVEGF145-2 (helper dependent viruses), are preferably administered to the host by direct injection into the area in and/or adjacent to ischemic tissue or quiescent tissue proximal to the area of ischemia, such as adipose or muscle tissue. It will of course be useful to transfect cells in the region of targeted adipose and muscle tissue. Transient expression of a VEGF₁₄₅ in these surrounding cells will result in a local extracellular increase in VEGF₁₄₅ and in turn will promote binding of recombinant VEGF to KDR to promote angiogenesis and in turn overcome the epoxic state associated with ischemia.

The recombinant first, second or helper-dependent AdVEGF₁₄₅ viruses of the present invention, including but not limited to AdVEGF145, AdHDVEGF145-1 and AdHDVEGF145-2, are also preferably delivered by i.v. injection. A recombinant adenovirus delivered by i.v. injection will preferentially infect hepatocytes, where expression persists for approximately 3-4 weeks for a first generation vector and possibly longer for helper dependent vector subsequent to the initial infection. Suitable titers will depend on a number of factors, such as the particular vector chosen, the host, strength of promoter used and the severity of the disease being treated. The skilled artisan may alter the titer of virus administered to the patient, depending upon the method of delivery, size of the tumor and efficiency of expression from the recombinant virus. A dose in the range of 10⁶-10¹¹ plaque forming units (pfus) is preferred to treat most tissue ischemias with VEGF₁₄₅ therapy. The skilled artisan will also realize that the number of viral particles encoding the transgene, whether or not replication competent in a complementing host cell, are a relevant dosing unit. In most adenovirus constructs, there are 50 to 100-fold more DNA containing particles than pfus.

There are many embodiments of the instant invention which those skilled in the art can appreciate from the specification. To this end, different transcriptional promoters, terminators, carrier vectors or specific gene sequences may be used successfully. Optimal precision in achieving concentrations of expressed VEGF₁₄₅ within the range that yields optimal efficacy requires a regimen based on the kinetics of the proteins availability to appropriate membrane receptor kinases. This involves a consideration of the strength of expression from the VEGF₁₄₅ construct, distribution, equilibrium, and elimination of the protein.

The present invention provides methods of gene therapy which stimulate angiogenesis in and adjacent to ischemic tissue in a mammalian host, preferably a human host. It will be readily apparent to the skilled artisan that various forms of the nucleotide sequence(s) encoding human or any mutated version thereof may be utilized to alter the amino acid sequence of the expressed protein. The altered expressed protein may have an altered amino acid sequence, yet still bind to KDR and in turn promote angiogenesis. For example, it is preferred that expressed protein lack the entire signal sequence, that is that wild type proteolytic processing of the 26 amino acid signal sequence be complete. However, it is within the scope of the invention that the leader sequence need not comprise the entire initial 26 amino acids of SEQ ID NO:2. In other words, the important point is that the final, mature product retain the ability to bind KDR and promote a mitogenic signal.

In an additional embodiment of the present invention a VEGF₁₄₅ protein or biologically active fragment thereof may be utilized to treat various peripheral and/or cardiac ischemias in the mammalian host. As an example but not forwarded as a limitation, recombinant human VEGF₁₄₅ as exemplified within this specification may be delivered from slow release polymers or devices into ischemic tissue or systemically. Pharmaceutically useful compositions comprising VEGF₁₄₅ can be formulated according to known methods such as by the admixture of a pharmaceutically acceptable carrier. Examples of such carriers and methods of formulation can be found in Remington's Pharmaceutical Sciences. To form a pharmaceutically acceptable composition suitable for effective administration, such compositions will contain an effective amount of the VEGF₁₄₅ protein, preferably recombinant human VEGF₁₄₅ protein.

Therapeutic or diagnostic proteinaceous compositions of the invention are administered to an individual in amounts sufficient to treat or diagnose disorders. The effective amount can vary according to a variety of factors such as the individual's condition, weight, sex and age. Other factors include the mode of administration. The pharmaceutical compositions can be provided to the individual by a variety of routes such as subcutaneous, topical, oral and intramuscular. The term “chemical derivative” describes a molecule that contains additional chemical moieties which are not normally a part of VEGF₁₄₅. Such moieties can improve the solubility, half-life, absorption, etc. of the protein. Examples of such moieties are described in a variety of texts, such as Remington's Pharmaceutical Sciences. Compounds identified according to the methods disclosed herein can be used alone at appropriate dosages. Alternatively, co-administration or sequential administration of other agents can be desirable.

The present invention also provides a means to obtain suitable topical, oral, systemic and parenteral proteinaceous pharmaceutical formulations for use in the methods of treatment of the present invention. The compositions containing compounds or molecules identified according to this invention as the active ingredient can be administered in a wide variety of therapeutic dosage forms in conventional vehicles for administration. For example, the compounds can be administered in such oral dosage forms as tablets, capsules (each including timed release and sustained release formulations), pills, powders, granules, elixirs, tinctures, solutions, suspensions, syrups and emulsions, or by injection. Likewise, they can also be administered in intravenous (both bolus and infusion), intraperitoneal, subcutaneous, topical with or without occlusion, or intramuscular form, all using forms well known to those of ordinary skill in the pharmaceutical arts.

Advantageously, compounds of the present invention can be administered in a single daily dose, or the total daily dosage can be administered in divided doses of two, three or four times daily. Furthermore, compounds for the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen.

For combination treatment with more than one active agent, where the active agents are in separate dosage formulations, the active agents can be administered concurrently, or they each can be administered at separately staggered times.

The dosage regimen utilizing the compounds of the present invention is selected in accordance with a variety of factors including type, species, age, weight, sex and medical condition of the patient; the severity of the condition to be treated; the route of administration; the renal, hepatic and cardiovascular function of the patient; and the particular compound thereof employed. A physician or veterinarian of ordinary skill can readily determine and prescribe the effective amount of the drug required to prevent, counter or arrest the progress of the condition. Optimal precision in achieving concentrations of VEGF₁₄₅ within the range that yields optimal efficacy requires a regimen based on the kinetics of the proteins availability to appropriate membrane receptor kinases. This involves a consideration of the distribution, equilibrium, and elimination of the protein.

The following examples are provided to illustrate the present invention without, however, limiting the same hereto.

### EXAMPLE 1

### Isolation of a cDNA Encoding Human VEGF₁₄₅

cDNA synthesis and PCR amplification—Five μg of human placenta total RNA (Clonetech Laboratories Inc. Cat #64024-1) was used for the first strand cDNA synthesis in 20 μl reaction using SuperScript pre-amplification system (GIBCO BRL Life Technologies Cat. #18089-011) for first strand cDNA synthesis. The reaction was primed with random hexamers. Following the synthesis reaction aliquots (2 μl) were used as templates in polymerase chain reactions using Pfu DNA polymerase. The following primers were used for the amplification of human VEGFs: forward primer, 5′-ACGGGATCCAAATATGAACTTTCTGCTCTCTTG-3′ (SEQ ID NO:3); reverse primer, 5′-TGGAAGCTTTCACCGCCTTGGCTTGTC-3′ (SEQ ID NO:4). PCR reactions were performed as follows; to each PCR reaction tube add 5 μl of 10×PCR reaction buffer (10×PCR reaction buffer is 100 mM KCl, 100 mM (NH₄)₂SO₄, 200 mM Tris-HCl, pH 8.75, 20 mM MgCl₂, 2.0 mM dNTP, 1.0% Triton X-100, 1 mg/ml bovine serum albumin [BSA]), 50 pmol of each primer and 2.5 units of Pfu DNA polymerase (Stratagene Cat. #600153) in a total volume of 50 μl. The reaction was first denatured for 5 min at 95° C., followed by 3 cycles of [2 min at 94° C., 1.5 min at 54° C., and 2 min at 72° C.,] then 27 cycles of [1.5 min at 94° C., 1 min at 68° C., 1.5 min 72° C.] and finally 10 min at 72° C. Two μl of the above reaction mixture was used as a template for a second round of PCR as follows; 5 min at 95° C., then 25 cycles of [1.5 min at 94° C., 1 min at 68° C., 1.5 min at 72° C.] followed by 10 min at 72° C.

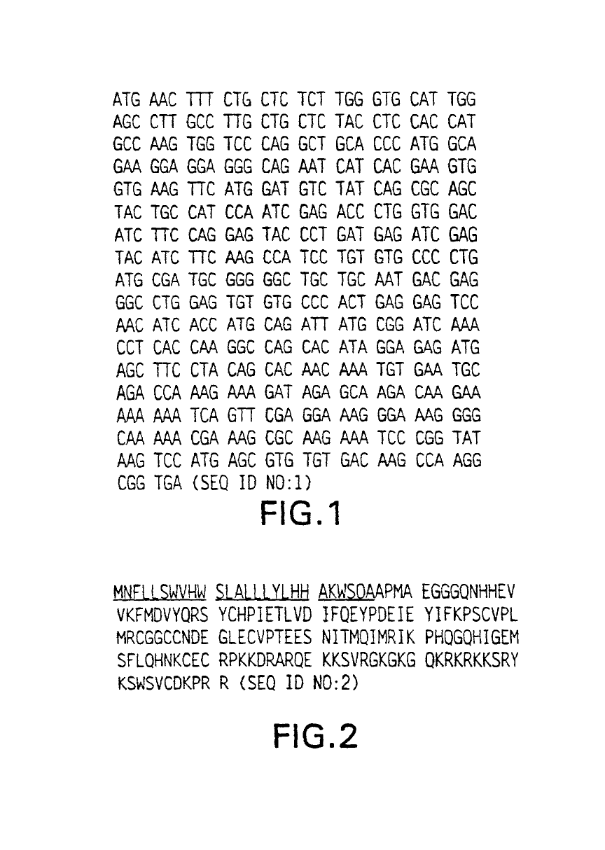

VEGF₁₄₅ cloning and confirmation—Products from the second round of PCR were visualized by gel electrophoresis on a 1% agarose gel. DNA bands corresponding to the expected size of the human VEGF isoforms 121 and 165 were amplified using either HeLa cell or placental RNA. In addition, a band corresponding to the expected size of VEGF₁₄₅ was detected only in the placental RNA reaction. The appropriate size band was excised, purified and subcloned into pCR-blunt plasmid using the Zero blunt PCR cloning kit (Invitrogen Cat. #K2700-20). The plasmid was transfected into competent E. coli cells supplied with the kit and cDNAs generated from colonies selected for kanamycin resistance were digested with Eco RI then analyzed by gel electrophoresis. Clones with the insert size expected for a human VEGF₁₄₅ gene fragment were confirmed by DNA sequence analysis on an ABI 377 automatic sequencer. A VEGF₁₄₅ DNA molecule which encodes human VEGF145 is shown in FIG. 1 and is set forth as SEQ ID NO:1. The deduced amino acid sequence human VEGF₁₄₅ is shown in FIG. 2 and set forth as SEQ ID NO:2.

### EXAMPLE 2

### Expression and Purification of Recombinant Human VEGF₁₄₅

Baculovirus expression of hVEGF₁₄₅—The hVEGF₁₄₅ gene fragment was isolated as a Bam HI/Hind III fragment and subsequently subcloned into the baculovirus expression vector pBlueBac4 (Invitrogen Cat. #V1995-20). The plasmid hVEGF₁₄₅/pBB4 was transfected into Sf21 cells using Bac-N-Blue transfection kit (Invitrogen Cat. #K855-01). Recombinant virus was isolated by plaque purification and the virus stock was expanded by 3 rounds of infection at a multiplicity of infection (MOI) of 0.1 pfu/cell. Protein was produced by infecting Sf21 cells at an MOI of 5 pfu/cell at a cell density of 1.5×10⁵ cell/ml in HyQ serum-free medium (Hyclone Cat #SH30065.02). The infection was incubated at 27° C. for 72 hr with constant stirring and the medium was harvested by centrifugation (1000×g for 10 min).

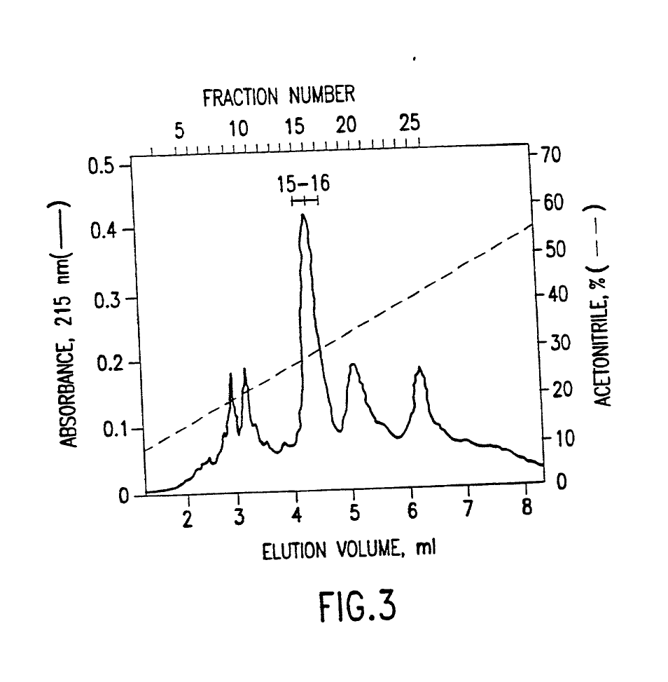

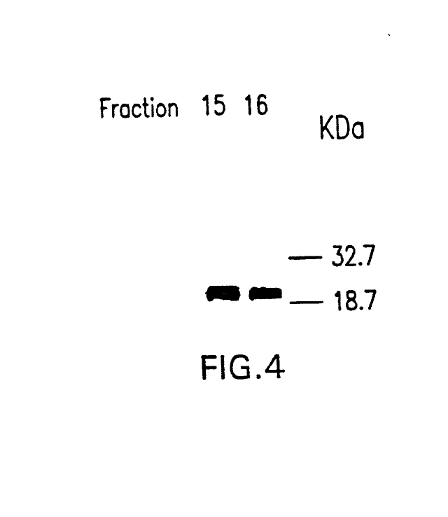

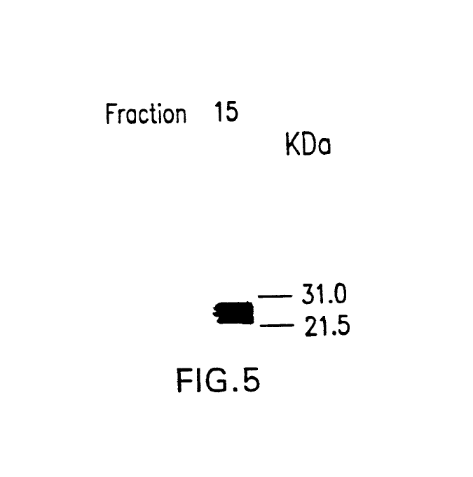

Purification of recombinant VEGF₁₄₅—The concentration of hVEGF₁₄₅ was determined by a hVEGF ELISA (R & D Systems,Cat #DVE00) according to the manufacture's instruction using hVEGF₁₆₅ (Cat #293-VE-010) supplied with the kit as a concentration standard. Typical hVEGF₁₄₅ expression levels were 200-400 μg/l of infected cells. Conditioned medium containing recombinant hVEGF₁₄₅ was directly loaded onto a 1 ml heparin-Sepharose column (Pharmacia Cat. #17-0406-01) equilibrated with phosphate buffer saline (PBS), pH 7.2. The column was washed with PBS buffer containing 0.4 M NaCl, followed by a step elution with the same buffer containing 0.8 M NaCl. Fractions were analyzed for VEGF by SDS/PAGE followed by Western blotting using a polyclonal antibody (MSD88) raised against recombinant human VEGF165. Peak fractions containing hVEGF₁₄₅ were pooled, diluted 16-fold with H₂O, and then loaded onto a Mono S HR5/5 column (Pharmacia Cat. #17-0547-01). The column was eluted with a linear gradient (0-100%) from 0.5×PBS, pH 7.2 to 0.8 M NaCl in PBS, pH 7.2 at a flow rate of 0.5 ml/min. Peak fractions were pooled and loaded onto a 4.6 mm×5 cm C4 column (Vydac Cat #214TP5405) then eluted with a 0-100% linear gradient (1%/min) from 0.1% trifluoroacetic acid to 70% acetonitrile containing 0.1% trifluoroacetic acid at a flow rate of 200 μl/min (FIG. 3). VEGF₁₄₅ was identified by Western blot (FIG. 4) and the purity was determined by separation on a 4-20% SDS/PAGE gel then visualized by silver stain (FIG. 5). Closely spaced bands most likely represent microhetergenous forms often times associated with baculovirus expression systems.

### EXAMPLE 3

### Biological Activity of Human VEGF₁₄₅

Human Umbilical Vein Endothelial Cell Mitogenesis Assay—Expression of VEGF mitogenic receptors that mediate mitogenic responses to the growth factor is largely restricted to vascular endothelial cells. Human umbilical vein endothelial cells (HUVECs) in culture proliferate in response to VEGF treatment and can be used as an assay system to quantify the effects VEGF isoforms. In the assay described in this Example Section, quiescent HUVEC monolayers are stimulated to proliferate with VEGF. The mitogenic response as a function of VEGF is determined by measuring the incorporation of [³H]thymidine into cellular DNA.

Methods—HUVECs frozen as primary culture isolates are obtained from Clonetics Corp. Cells are maintained in Endothelial Growth Medium (EGM; Clonetics) and are used for mitogenic assays at passages 3-7. Monolayers maintained in EGM are harvested by trypsinization and plated at a density of 4000 cells per 100 μl Assay Medium per well in 96-well plates (NUNCLON 96-well polystyrene tissue culture plates [NUNC #167008]). Cells are growth-arrested for 24 hours at 37° C. in a humidified atmosphere containing 5% CO₂. After the 24-hour quiescent period, 10 μl/well of Assay Medium (Dulbecco's modification of Eagle's medium containing 1 mg/ml glucose [low-glucose DMEM; Mediatech] plus 10% (v/v) fetal bovine serum [Clonetics]) containing 10×VEGF solutions are added over a concentration range spanning the mitogenic dose/response curve. Cells are then incubated at 37° C./5% CO₂. Solutions of purified human VEGF₁₆₅ (500 ng/ml; R&D Systems, expressed in Sf21 cells) and purified human VEGF₁₄₅ were prepared in Assay Medium. Concentrations of VEGF isoforms were determined by using an enzyme-linked immunosorbent assay (R&D systems). After 24 hours in the presence of growth factors, 10×[³H]Thymidine (10 μl/well) is added. 10×[³H]Thymidine is [Methyl-³H]Thymidine (20 Ci/mmol; Dupont-NEN), diluted to 80 μCi/ml in low-glucose DMEM. Three days after addition of [³H]thymidine, medium was removed by aspiration, and cells are washed twice with Cell Wash Medium (400 μl/well followed by 200 μl/well). Cell Wash medium was Hank's balanced salt solution (Mediatech) containing 1 mg/ml bovine serum albumin (Boehringer-Mannheim). The washed, adherent cells are then solubilized by addition of Cell Lysis Solution (100 μl/well) and warming to 37° C. for 30 minutes. Cell Lysis Solution is 1 N NaOH, 2% (w/v) Na₂CO₃. Cell lysates are transferred to 7 ml glass scintillation vials containing 150 μl of water. Scintillation cocktail (5 ml/vial) is added, and cell-associated radioactivity is determined by liquid scintillation spectroscopy.

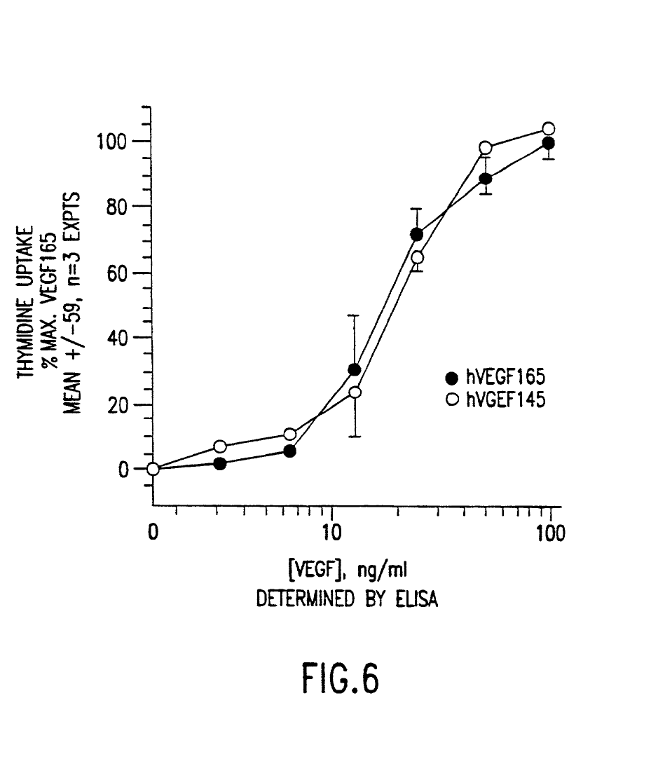

Results—Purified baculovirus-expressed human VEGF₁₄₅ was fully efficacious as an endothelial cell mitogen when compared to baculovirus-expressed human VEGF₁₆₅ (FIG. 6) with equivalent half-maximal activities of 17-19 ng/ml for VEGF165 and VEGF₁₄₅.

### EXAMPLE 4

### Construction of the First Generation Adenovirus Vector ADVEGF145

Several systems have been developed for the construction of first generation adenovirus vectors and have been recently reviewed by Graham and Prevec (1995, Mol. Biotech. 3: 207-220) and Hitt et al. (1995, Techniques for human adenovirus vector construction and characterization, In Methods in Molecular Genetics, Volume 7. Molecular Virology Techniques Part B, ed. Kenneth W. Adolph, Academic Press, Inc. Orlando, Florida). All of these systems involve cloning the transgene of interest (coding region flanked by appropriate regulatory sequences) into a shuttle plasmid in which it is flanked by Ad sequences homologous to the region of the viral genome into which the transgene will be introduced. The transgene is then rescued into virus by either direct ligation in vitro followed by transfection into 293 cells, homologous recombination in bacteria followed by transfection into 293 cells (Chartier et al., 1996, Journal of Virology 70: 4805-4810), or by in vivo homologous recombination following transfection into 293 cells.

E1 shuttle plasmids have been developed for the rescue of inserts into the E1 region. These plasmids contain the left 16% of the Ad genome with a deletion of E1 sequences and cloning sites into which the transgene is introduced. If convenient restriction sites are available in the vector backbone, direct ligation of the shuttle plasmid to purified viral DNA can be performed in vitro followed by transfection into 293 cells to generate infectious virus. This method although efficient can require extensive screening if the viral DNA is not completely restricted and in many cases is not practical due to the lack of unique correctly positioned restriction sites. For these reasons many protocols rely on in vivo homologous recombination to generate infectious virus.

To construct a virus by homologous recombination in 293 cells the E1 shuttle plasmid can be transfected into 293 cells with purified viral DNA that has been restricted in the left end or with viral DNA contained in a second plasmid (an Ad genome plasmid). As with direct ligation the use of purified viral DNA sometimes requires extensive screening to obtain the desired vector because of the regeneration of parental virus and for this reason plasmid systems are more desirable. A number of Ad genome plasmid systems have been developed for rescuing inserts into E1 (McGrory et al., 1988, Virology 163: 614-6170) or E3 (Ghosh-Choudhury, et al., 1986, Gene 50: 161-171; Mittal, et al., 1993, Virus Res. 28: 67-90) or both (Bett et al., 1994, Proc. Natl. Acad. Sci. USA 91: 8802-8806) regions.

To construct a virus by homologous recombination in E. coli a segment of the E1 shuttle plasmid containing the transgene flanked by adenoviral sequences is gel purified and used to transform E. coli along with an Ad genome plasmid which has been linearized in the region in which the transgene is to be rescued. Homologous recombination between the two DNA's results in a repaired plasmid which can then be selected, grown up and purified from the bacteria and used to transfect 293 cells to generate virus.

To construct the first generation vector expressing VEGF145 the system involving homologous recombination in E. coli was used (Chartier et al., 1996, Journal of Virology, 70: 4805-4810). The steps involved in the construction are outlined below. The coding sequences for VEGF₁₄₅ were obtained from the pCR-blunt clone described above by digestion with BamHI and EcoRI and cloned into the E1 shuttle plasmid pHCMVI1BGHpA-2, generating pHCMVI1VEGF145. To remove an undesirable PacI restriction site pHCMVI1VEGF145 was digested with PacI, treated with T4 DNA polymerase and religated, generating pHCMVI1VEGF145P-. pHCMVI1VEGF145P- was then digested with SspI and Bst1107I and the fragment containing the transgene flanked by Ad sequences was gel purified. The purified fragment was then used to transform E. coli strain BJ5183 along with Ad genome plasmid pHVAd1 that was linearized in the E1 region by ClaI digestion. PHVAd1 contains the entire Ad genome with a deletion of E3 sequences from Ad bp 28133 to bp308180 and has the viral ITR's separated by plasmid sequences which contain the Ampicillin resistance gene and bacterial origin of replication. Homologous recombination between the purified shuttle plasmid fragment and linearized pHVAD1 generated a repaired plasmid designated pHVAdVEGF145P-. Bacterial transformants carrying pHVAdVEGF145P- were isolated and the plasmid DNA extracted and used to transform E. coli strain HB101 in which the plasmid grows more efficiently. pHVAdVEGF145P- plasmid DNA extracted and purified from HB101 cultures was digested with PacI to liberate the viral ITR's from plasmid DNA sequences and used to transfect 293 cells. The virus AdVEGF145 was obtained from this transfection.

### EXAMPLE 5

### Construction of the Helper Dependent Adenovirus Vectors ADHDVEGF145-1 and ADHDVEGF145-2

Helper-dependent Ad vectors are deleted of all viral coding sequences and contain only the cis acting viral sequences needed for DNA replication (the ITR's 1-103 bp located at each end of the genome) and genome encapsidation (packaging signals 194-358 bp). The helper-dependent vector carries the transgene and “stuffer” DNA (noncoding DNA) required to generate a vector that is efficiently packaged. For efficient packaging the vector genome should not be less than 75% (approximately 28 Kb) and the upper limit not more than 105% (approximately 38 Kb) of the wt Ad genome size of 36 Kb. All other viral proteins are provided in trans from a helper virus.

The helper virus AdLC8cLUC is an E1-deleted first generation vector which contains lox P sites flanking its packaging signals. When 293 cells expressing the cre-recombinase are coinfected with the helper virus and dependent vector, the packaging signals are excised from the helper virus preventing it from being encapsidated, while allowing its genome to provide functions in trans to the dependent vector. Five to six serial passages are needed to increase the titer of the helper-dependent vector prior to a large-scale amplification from which vector is purified on cesium chloride gradients.

The steps involved in the construction of the helper-dependent Ad vectors expressing VEGF145 are outlined below. The methods for the construction of helper-dependent Ad vectors are described in Parks et al (1996, Proc. Natl. Acad. Sci. 93: 13565-13570). The coding sequences for VEGF145 were obtained from the pCR-blunt clone described above by digestion with BamHI and EcoRI and cloning into the plasmid expression vector pV1Jns (described above and in PCT International Application WO97/31115), generating pV1JnsVEGF145. The transgene cassette was then removed from pV1JnsVEGF145 by digestion with SfiI and MscI, treated with T4 DNA polymerase to generate blunt ends and cloned into the HindII site in helper dependent shuttle plasmid pABSHD-3, generating pSHDVEGF145-1 and pSHDVEGF145-2. Helper-dependent shuttle plasmid pABSHD-3 contains a multiple cloning region adjacent to a kanamycin resistance gene that allows for the selection of the desired recombinant plasmid after cloning into the ampicillin resistance gene containing helper-dependent backbone plasmid pSTKI20. The transgene/Kan cassette was removed from pSHDVEGF145-2 by FseI digestion and cloned into pSTK120 generating pSTKVEGF145Kan-1 and pSTKVEGF145Kan-2. Finally, the kanamycin resistance gene was removed from both pSTKVEGF145Kan-1 and pSTKVEGF145Kan-2 by digestion with AscI followed by ligation, generating pSTKVEGF145-1 and pSTKVEGF145-2 respectively. pSTKVEGF145-1 and pSTKVEGF145-2 were then digested with PmeI to release the viral ITR's from plasmid sequences and transfected into 293 cells, which were infected with helper virus AdLC8cLUC 24 hours later. When the cells were completely lysed the medium was collected and used to infect 293cre4 cells. Five serial passages in 293cre4 cells were required to increase the titer of the helper dependent vectors prior to large-scale vector purification.

What is claimed:

1. A method of stimulating angiogenesis in a mammalian host which comprises delivering a DNA vector to said mammalian host, said DNA vector expressing VEGF145 or a biologically active fragment thereof.

2. The method of claim 1 wherein said mammalian host is a human.

3. The method of claim 2 wherein said DNA vector is a recombinant adenovirus.

4. The method of claim 2 wherein said DNA vector is a recombinant DNA plasmid vector.

5. The method of claim 3 wherein said recombinant adenovirus is delivered by infection into cells within or adjacent to a tissue ischemia.

6. A method of stimulating angiogenesis in a mammalian host which comprises delivering a DNA vector to said mammalian host, said DNA vector expressing human VEGF145 as set forth in SEQ ID NO:1, or a biologically active fragment thereof.

7. The method of claim 6 wherein said mammalian host is a human.

8. The method of claim 7 wherein said DNA vector is a recombinant adenovirus.

9. The method of claim 7 wherein said DNA vector is a recombinant DNA plasmid vector.

10. The method of claim 8 wherein said recombinant DNA plasmid vector is delivered by injection into cells within or adjacent to a tissue ischemia.

11. The method of claim 8 wherein said recombinant adenovirus is delivered by infection into cells within or adjacent to an ischemic peripheral or cardiac tissue.

12. The method of claim 11 wherein said recombinant adenovirus is AdVEGF145.

13. The method of claim 11 wherein said recombinant adenovirus is AdHDVEGF145-1.

14. The method of claim 11 wherein said recombinant adenovirus is AdHDVEGF145-2.

15. The method of claim 9 wherein said recombinant DNA plasmid vector is delivered by injection into cells within or adjacent to a tissue ischemia.

16. The method of claim 9 wherein said recombinant adenovirus is delivered by infection into cells within or adjacent to an ischemic peripheral or cardiac tissue.

17. The method of claim 16 wherein said recombinant DNA plasmid vector is pV1JnsVEGF145.

18. A recombinant virus comprising a DNA fragment encoding humanVEGF₁₄₅ containing at least one regulatory sequence which controls expression of said DNA fragment within a mammalian host.

19. A recombinant virus of claim 18 which is a recombinant adenovirus.

20. A recombinant adenovirus of claim 19 wherein said DNA fragment encodes a human VEGF₁₄₅ as set forth in SEQ ID NO:2.

21. A recombinant adenovirus of claim 20 selected from the group consisting of AdVEGF-145, AdVEGF145-1 and AdVEGF145-2.


SEQUENCE LISTING (5 sequences)

SEQ ID NO: 1 - DNA, 516 bp, Human
atgaactttctgctctcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtgtgacaagccaaggcggtga

SEQ ID NO: 2 - protein, 171 aa, Human
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVCDKPRR

SEQ ID NO: 3 - DNA, 33 bp, Artificial Sequence
  oligonucleotide
acgggatccaaatatgaactttctgctctcttg

SEQ ID NO: 4 - DNA, 27 bp, Artificial Sequence
  oligonucleotide
tggaagctttcaccgccttggcttgtc

SEQ ID NO: 5 - DNA, 4864 bp, Artificial Sequence
  E. coli
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc